(19) United States
(12) Patent Application Publication
(10) Pub. No.: US 2022/0039804 A1
(43) Pub. Date: Feb. 10, 2022

(54) FLOW-DIVERTING IMPLANT AND DELIVERY METHOD

(71) Applicant: MicroVention, Inc., Aliso Viejo, CA (US)

(72) Inventors: Hussain S. Rangwala, Aliso Viejo, CA (US); Ronak Dholakia, Aliso Viejo, CA (US)

(73) Assignee: MicroVention, Inc., Aliso Viejo, CA (US)

(21) Appl. No.: 17/393,306

(22) Filed: Aug. 3, 2021

Related U.S. Application Data
(60) Provisional application No. 63/061,099, filed on Aug. 4, 2020.

(51) Int. Cl. A61B 17/12; A61F 2/06

(52) U.S. Cl. CPC A61B 17/12113; A61F 2/06; A61B 17/12031; A61F 2220/0008; A61F 2002/068; A61B 2017/12068; A61F 2230/0095; A61B 17/12145

(57) ABSTRACT

A flow-diverting implant includes a saddle-shaped braided mesh diverter that is sized to provide adequate blocking coverage of a neck of an aneurysm. The diverter is anchored using minimally profiled, generally circular anchors that present little to no resistance to blood flow and are unlikely to create thrombosis. A locator extends into the aneurysm to ensure the diverter is optimally positioned over the neck of the aneurysm.

### RELATED APPLICATIONS

This application claims benefit of and priority to U.S. Provisional application Ser. No. 63/061,099 filed Aug. 4, 2020 entitled FLOW DIVERTER, which is hereby incorporated herein by reference in its entirety.

### BACKGROUND OF THE INVENTION

Weakened vascular regions can lead to aneurysms. An aneurysm is an abnormal bulging of a blood vessel wall. The vessel from which the aneurysm protrudes is the parent vessel. Saccular or sidewall aneurysms look like a sac protruding out from the parent vessel. Saccular aneurysms have a neck and can be prone to rupture. Fusiform aneurysms are a form of aneurysm in which a blood vessel is expanded circumferentially in all directions. Fusiform aneurysms generally do not have a neck and are less prone to rupturing than saccular aneurysms. As an aneurysm grows larger, its walls generally become thinner and weaker due to the high pulsatile pressures associated with blood. This decrease in wall integrity, particularly for saccular aneurysms, increases the risk of the aneurysm rupturing and hemorrhaging blood into the surrounding tissue, with serious and potentially fatal health outcomes if the aneurysms are left untreated.

Traditional treatments of aneurysms involve, for example, occlusion or clipping. Occlusion involves filling the aneurysm with an occlusive device, such as coils, to occlude the blood flow into the aneurysm and cut off blood supply to the aneurysm over time. Clipping to treat aneurysms involves placing a small metal clip on the neck of the aneurysm to obstruct the flow of blood into the aneurysm.

Recent techniques in aneurysm treatment have utilized flow diversion stents, which are braided tubular devices placed across a substantial length inside the vessel covering the neck of an aneurysm. A flow diversion stent (also known as flow diverters) utilize a reduced porosity interface to limit blood flow into the aneurysm. This stent causes flow stagnation within the aneurysm and occlusion of the aneurysm thereby limiting the risk of rupture. Eventually, tissue grows over the flow diversion stent, and seals the aneurysm.

Traditional flow diverters that utilize fixed tubular or cylindrical stent structures with uniform low porosity over an entire length and circumference of the stents have several drawbacks. For example, the implantation of a uniformly-covered, low porosity tubular stent necessarily results in covering healthy walls of the blood vessel, thereby blocking the blood flow to the covered regions. Doing so may cause thrombosis or blood clots in non-diseased areas inside the vessel. Furthermore, due to the significant surface area of these tubular flow diverters, the current aneurysm treatment protocol requires the use of aggressive dual antiplatelet therapy medication for at least 6 months to prevent the stent-related thrombosis/blood clots.

Another drawback associated with the typical tubular flow diversion stent is observed when the aneurysm is positioned near a vessel bifurcation. The consistent low-porosity tubular interface over the entire length and breadth of the stent may overlap at the bifurcation with other non-diseased blood vessels which are not affected by aneurysms and thereby negatively restricts blood flow into these vessels.

Hence, to address at least the above issues, there is a need for a flow-diverting stent which utilizes a flow diverter that can selectively be placed in contact with the neck of the aneurysm and does not impact the blood flow around the non-diseased regions of the vessel or with other non-diseased blood vessels which are not affected by the aneurysm.

There is a further need for a flow-diverting stent design that minimizes an amount of material encountered by blood flowing through the vessel. Such a stent design must not sacrifice anchor strength as a tradeoff for reduced material.

### OBJECTS AND SUMMARY OF THE INVENTION

In some embodiments, a flow-diverting implant to reduce the blood flow inside an aneurysm is described.

In one embodiment, the flow-diverting implant comprises a flow diverter which may be braided. The flow-diverting implant further comprises a first anchor and a second anchor and a locator unit. According to some embodiments, the flow-diverting implant is positioned properly when the locator unit is placed through the neck of the aneurysm. The flow diverter further comprises of dense braided mesh with small pores characterized by pore density of between 120-170 picks per inch, 10-20 pores/mm² and device porosity between 60-70% to perform it's flow diversion function and stagnate blood flow inside the aneurysm. The braided mesh can exhibit wire diameters ranging from 0.00075 inch to 0.00175 inch and hybrid pattern utilizing wires of different diameters.

In some embodiments, the flow diverter, which restricts the blood flow inside the aneurysm, is positioned only along a particular radial portion of the flow-diverting implant and the flow diverter aligns selectively with the neck of the aneurysm to selectively block blood flow inside the aneurysm.

In some embodiments, the first anchor of the flow-diverting implant is positioned at the proximal portion of the flow diverter and the second anchor is positioned at the distal portion of the flow diverter.

In some embodiments, the locator unit is positioned about a middle portion of the flow diverter and the locator unit expands inside the aneurysm.

In some embodiments, the locator unit positioned about the middle portion of the flow diverter aligns the flow diverter with the neck of the aneurysm when the locator unit expands inside the aneurysm. The first anchor may be positioned at the proximal portion of the flow diverter and the second anchor may be positioned at the distal portion of the flow diverter, and they may expand in a direction substantially opposite the locator unit to anchor the flow-diverting implant inside a blood vessel.

According to some embodiments, the first anchor, the second anchor and the locator comprise wires having circular, coiled or spiral shapes. In some embodiments, the first anchor and the second anchor diagonally connect a front edge of the flow diverter to a back edge of the flow diverter. In some other embodiments, the first anchor and the second anchor longitudinally connect a proximal portion of the flow diverter to a distal portion of the flow diverter. In some embodiments, the locator unit diagonally connects a front edge of the flow diverter to a back edge of the flow diverter. In yet some other embodiments, the locator unit longitudinally connects a proximal portion of the flow diverter to a distal portion of the flow diverter.

In some embodiments, the flow diverter comprises a shape memory material such as nitinol, for example. In some embodiments, the flow diverter comprising the shape memory material is functionalized with poly(MEA-c-APMA) to impart hydrophilic properties to the surface. The functionalization of Nitinol and other shape memory alloys with poly(MEA-co-APMA) is described in U.S. Pat. No. 10,543,299 to Baldwin et al. entitled, “SURFACE COATINGS,” U.S. Pat. No. 10,087,526 to Baldwin entitled, “STENTS, PACKAGING, AND SUBSTANCES USED WITH STENTS,” and U.S. Pat. Pub. No. 2018/0325649 to Wu et al. entitled, MEDICAL DEVICES, all of which are hereby incorporated by reference in their entirety.

One aspect of the invention is a method of blocking blood flow to an aneurysm comprising: navigating a delivery pusher containing an implant to a neck of an aneurysm; advancing the implant out of the delivery pusher such that a locator unit of the implant expands into the aneurysm; allowing a remaining portion of the implant to expand such that a braided portion of the implant blocks a neck of the aneurysm and at least one anchor extends into a blood vessel from which the aneurysm formed; and, releasing the implant.

In some embodiments, advancing the implant out of the delivery pusher such that the locator unit of the implant expands into the aneurysm comprises rotating the pusher until the locator aligns with the neck of the aneurysm.

In some embodiments, positioning the flow diverter at the neck of the aneurysm further comprises rotating the pusher back and forth to place the locator inside the aneurysm and expanding the locator radially inside the aneurysm and anchoring the at least one anchor inside the blood vessel.

In at least one embodiment, releasing the implant comprises severing a tether connecting the implant to the delivery pusher.

In at least one embodiment, severing the tether comprises activating a heater in operable proximity to the tether.

One aspect of the invention is a flow diverting implant comprising: a flow diverter; a first anchor positioned at a proximal portion of the flow diverter; a second anchor positioned at a distal portion of the flow diverter; and a locator unit configured such that when in a deployed state for reception in the aneurysm, the flow diverter is in an diverting state and the first and second anchors are in an anchoring state.

Another aspect of the invention is a flow diversion system comprising: a delivery pusher including: a proximal end and a distal end; a heating element proximal the distal end; a power source connected to the heating element with a switch; an activation mechanism near the proximal end that closes the switch; an implant comprising: a flow diverter having a proximal end; at least one anchor attached to the flow diverter; and a locator unit attached to the flow diverter; wherein the implant is connected to the delivery pusher with a tether that passes within operable proximity to the heating element such that when the switch is closed, the tether is heated and severed, thereby releasing the implant.

According to some embodiments, severing the tether comprises activating a heater in operable proximity to the tether. is detached from the pusher by severing a tether connecting between the proximal end of the flow diverter to the distal end of the pusher. In yet some embodiments, the flow diverter is detached from the pusher by severing the tether through thermal, mechanical or electrochemical detachments.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which:

FIG. 1 illustrates a side wall aneurysm inside a blood vessel;

FIG. 2 is a perspective view of a prior art conventional flow diversion stent to treat a sidewall aneurysm;

FIG. 3 is a perspective view of an embodiment of a flow diversion device with a targeted flow diverter of the invention;

FIG. 4 is an enlarged perspective view of the embodiment of FIG. 3;

FIG. 5 is a top plan view of an embodiment of the flow diversion device of FIGS. 3 and 4;

FIG. 6 is a perspective view of an embodiment of a flow diversion device of the invention;

FIG. 7 is an end view of an embodiment of a flow diversion device of the invention;

FIG. 8 is a perspective view of an embodiment of a flow diversion device of the invention;

FIG. 9 is side elevation of a flow diversion device/delivery pusher interface for delivering the flow diversion device inside the aneurysm;

FIG. 10 is a perspective view of an embodiment of a delivery device attached to an embodiment of an implant of the invention;

FIG. 11 is a cutaway view of an embodiment of a detachment mechanism of the invention;

FIG. 12 is a perspective view of an embodiment of an implant attached to an embodiment of a delivery device prior to detachment; and

FIG. 13 is a per perspective view of an embodiment of an implant just after being released from an embodiment of a delivery device.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

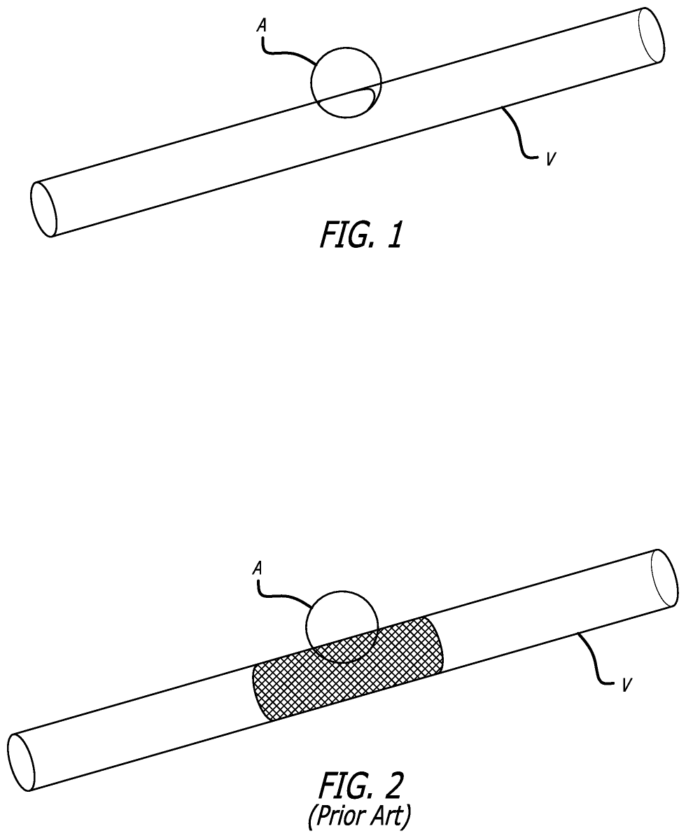

Referring now to the Figures, and first to FIG. 1, there is shown a saccular or sidewall aneurysm A in a blood vessel V. The aneurysm is prone to rupture if left untreated because it may have weakened due to high pulsatile pressure of blood.

A prior art flow-diverting stent implanted in the area surrounding the neck of the aneurysm is shown in FIG. 2. This typical traditional flow diverter utilizes a tubular lattice structure with a low porosity interface covering the entire length and circumference of the stent. It is apparent that while only the neck of the aneurysm needs to be blocked to inhibit blood flow, a majority of the low porosity material of the diverter is not blocking the neck. This extra material is prone to creating the issues described above.

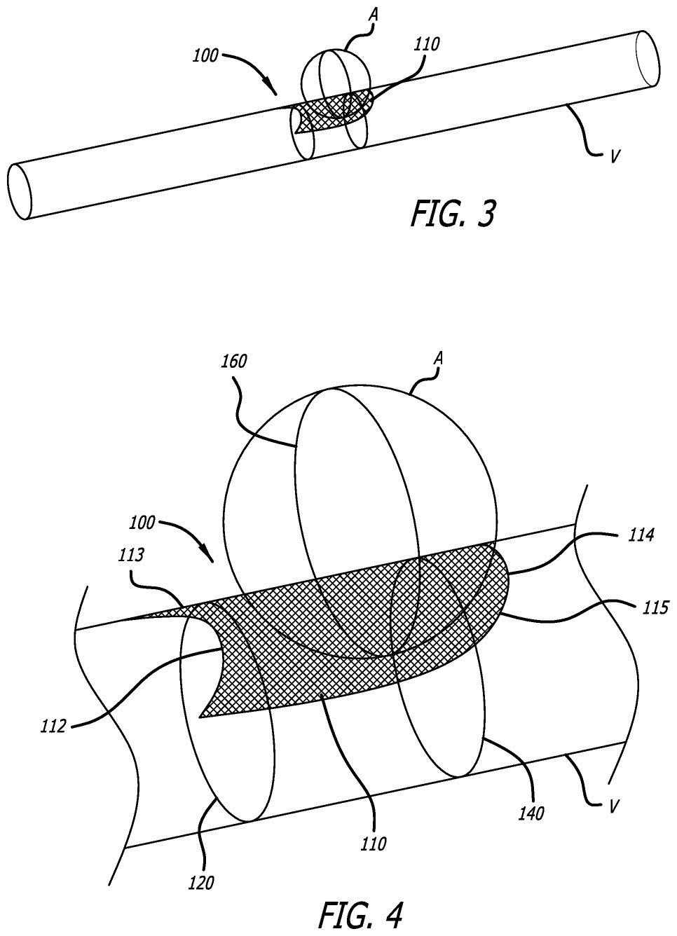

FIG. 3 shows one embodiment of an improved flow-diverting implant 100 that utilizes a reduced-area, low-porosity flow diverter 110. Because the device 100 positions a low-porosity interface only at the neck of the aneurysm, it reduces the chances of thrombosis in non-diseased areas inside the vessel. The reduced profile also prevents blocking flow to other vessels at bifurcations.

FIG. 4 illustrates an enlarged view of the flow-diverting implant 100 shown in FIG. 3. The flow-diverting implant 100 generally comprises a flow diverter 110, anchors 120 and 140, and a locator 160.

The flow diverter 110 is shaped to conform to the generally tubular shape of the vessel. Shaped edges 112 and 114 give the flow diverter 110 a saddle shape. The mesh of the flow diverter 110 may comprise wire, for example, a nitinol wire. The flow diverter 110 may include a metallic shape memory wires (e.g., nitinol, stainless steel, cobalt-chromium). Additionally or alternatively, drawn-filled tubing (DFT) utilizing a radiopaque (e.g., platinum or tantalum) core and an outer metallic (e.g., nitinol) jacket may be used. The braided mesh can exhibit wire diameters ranging from 0.00075 inch to 0.00175 inch and hybrid pattern utilizing wires of different diameters.

In at least one embodiment, the flow diverter 110 is woven from one wire braided to form a mesh having plurality of relatively small pores to block flow to the aneurysm and to promote cellular ingrowth. Alternatively, flow diverter 110 is woven from a plurality of wires, (e.g., between 2-64, 16-48 wires, or 24-32 wires).

The mesh is woven or braided to create small pores between the wires, characterized by pore density of between 120-170 picks per inch, 10-20 pores/mm² and device porosity between 60-70% to perform it's flow diversion function and to stagnate blood flow inside the aneurysm. The flow diverter 110 is placed adjacent to the neck of the aneurysm inside a vessel and the small pores of the flow diverter 110 obstruct blood flow within the aneurysm without affecting the blood flow to other non-diseased areas in the vessel.

Surface modification may be applied to the mesh to functionalize the device surface and characterize hydrophilic properties. For example, poly(MEA-c-APMA) is a coating that imparts hydrophilic properties. In some embodiments, the coating of the mesh is selected to encourage covalent bonding, for example, covalent bonding of the nitinol with the functional groups of the coated molecules. In some embodiments, the covalent bonding between the coating and the wire material of the mesh enables the coating to last longer on the mesh.

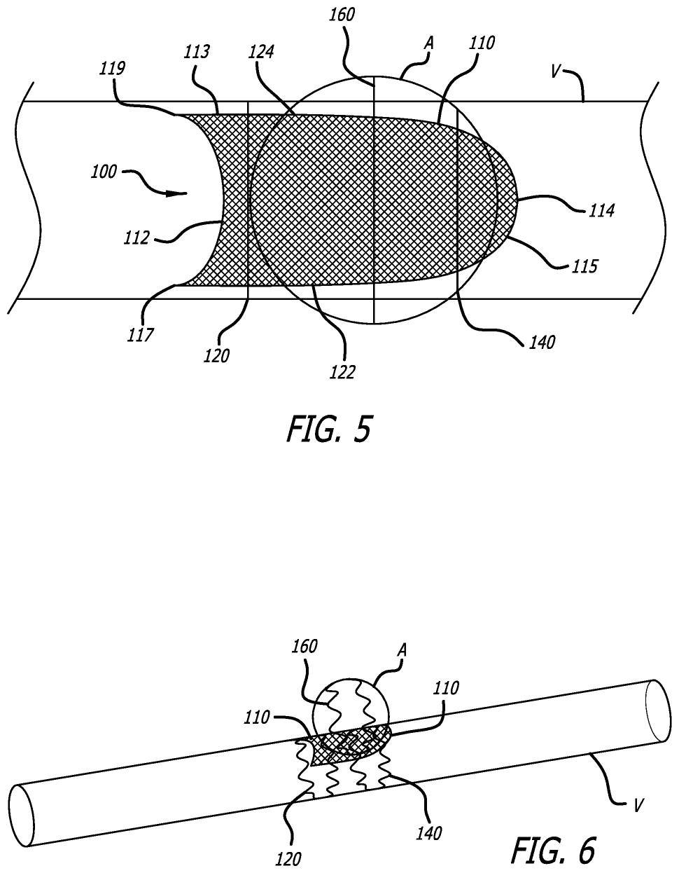

As can be further seen in FIG. 4, the flow-diverting implant 100 may include anchor units 120 and 140 to anchor the flow-diverting implant 100 inside the blood vessel, and to ensure forces placed on the flow diverter 110 are not transferred to the locator 160. In some embodiments, a proximal anchor 120 is located on a proximal portion of the flow diverter and may be in the form of a circular wire extending into the vessel when the flow diverter 110 is positioned at the neck of the aneurysm. In some embodiments, a distal anchor 140 is located on a distal portion 115 of the flow diverter and may be in the form of a circular wire extending into the vessel when the flow diverter 110 is positioned at the neck of the aneurysm. According to some embodiments, a proximal portion 113 of the flow diverter 110 is attached to the top portion of the proximal anchor 120 and a distal portion 115 of the flow diverter 110 is attached to the top portion of the distal anchor 140, as can be seen in FIGS. 4 and 5. However, the connections between the flow diverter 110 and the anchors may not be limited to this configuration. In some embodiments, each anchor may stretch from the proximal end 116 to the distal end 118 of the flow diverter 110, as shown in FIG. 8 and discussed in detail in later section of this application.

As can be further seen in FIG. 4, the flow diverter 110 may be of a braided mesh construction with small pores and sized such that the mesh does not sit around the entire circumferences of the proximal anchor 120 and distal anchor 140. Rather, the flow diverter 110 covers only, for example, small sections of the proximal anchor 120 and distal anchor 140. In some embodiments, when the flow-diverting implant 100 is deployed inside the vessel, the proximal anchor 120 and distal anchor 140 provide a scaffold for the flow diverter 110 and help anchor the flow-diverting implant 100 inside the vessel.

FIG. 4 further illustrates a third structural unit comprising a wire which may be placed radially inside the aneurysm A as an aneurysm locator 160. The locator 160 helps identify the orientation of the flow diverter 110 with respect to the neck of the aneurysm. In the present application, since the flow diverter 110 is only along a particular radial portion of the flow-diverting implant 100, the flow diverter 110 must be correctly aligned with the aneurysm neck. In this way, since the locator 160 is attached to the flow diverter 110, when the locator is aligned in the aneurysm, the user may confirm the proper placement of the flow diverter 110 with respect to the neck of the aneurysm.

In some embodiments of the present invention, the proximal anchor 120 and distal anchor 140 may be woven as single wires through the braided mesh of the flow diverter 110 and are positioned inside the vessel and include but are not limited to circular end shapes. According to some embodiments, the locator 160 may be woven as single wire through the braided mesh of the flow diverter 110 and is positioned inside the aneurysm and includes but is not limited to a circular shape.

FIG. 5 illustrates a plan view of the flow diverter 110 when it is positioned at the neck of the aneurysm. FIG. 5 also shows the positions of the proximal anchor 120 and distal anchor 140 when they are expanded inside the vessel and the position of the locator 160 when it is expanded inside the aneurysm. It is clear from FIGS. 4 and 5 that the circular end shape of the locator 160 is confined only inside the aneurysm and does not protrude into the vessel. Similarly, the proximal anchor 120 and distal anchor 140 are positioned inside the vessel and do not protrude into the neck of the aneurysm.

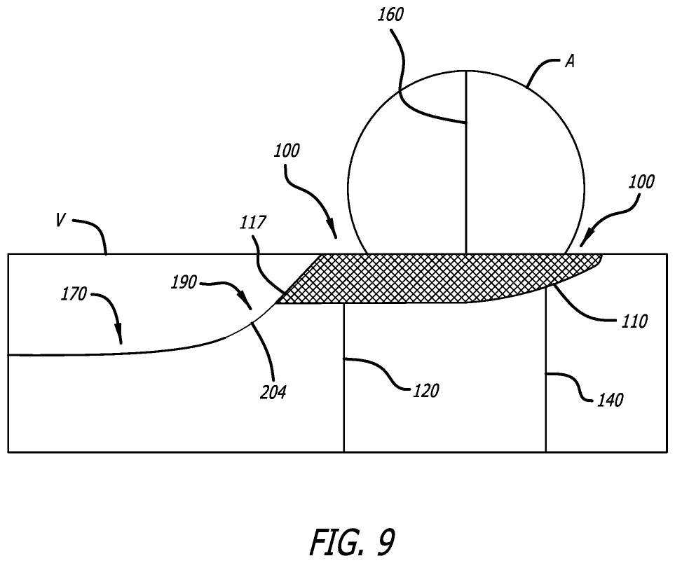

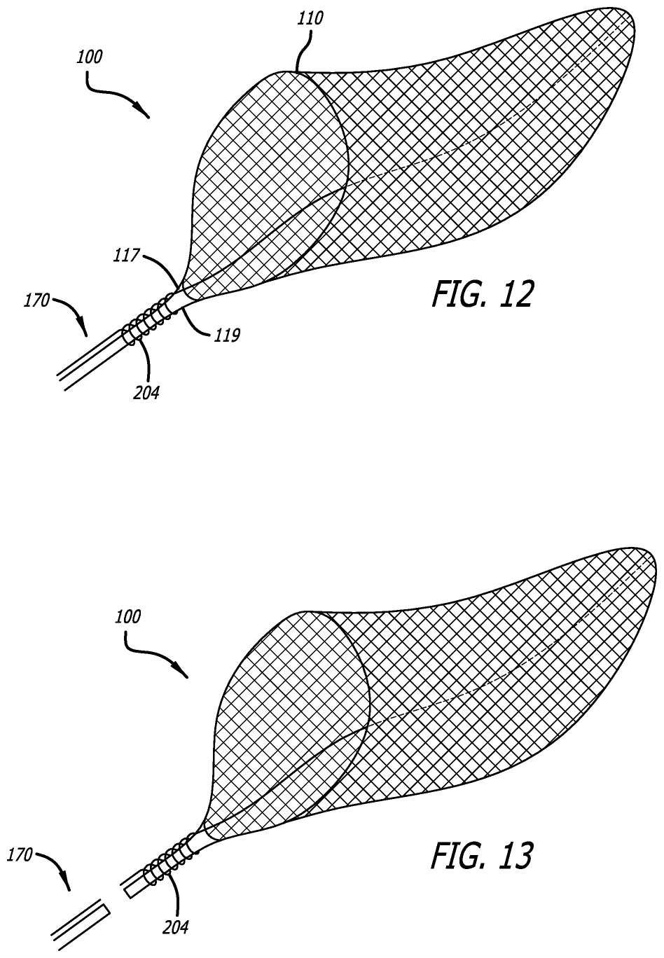

FIG. 5 further shows that the flow diverter 110 includes a shape of a saddle having a rounded distal portion 114 and a curved proximal portion having tapered corners 117, 119. The rounded distal portion 115 of the flow diverter reduces the drag force inside the blood vessel. The tapered corners 117, 119 are shaped to allow the implant to be tied to a delivery device with a tether 204 (as shown in FIGS. 9-13). Although, the implant could be tied to the delivery device using only one corner 117 or 119, using both corners to attach the implant to the delivery device assists in proper location and deployment. The tethered corners 117 and 119, being tapered, further allows a substantial expansion of the implant 100 prior to release to allow verification that the implant is properly seated, i.e., the flow diverter 110 is longitudinally positioned at the neck of the aneurysm such that the proximal portion 113 of the flow diverter 110 is positioned at one end of the neck of the aneurysm and the distal portion 115 of the flow diverter 110 is positioned at the other end of the neck of the aneurysm, as can be seen in FIGS. 4 and 5.

The shapes of the proximal anchor 120, distal anchor 140 and locator 160 may have other forms instead of circular. In some embodiments of the present invention, the proximal anchor 120, distal anchor 140 and the locator 160 may take the forms of coiled wires or spiral wires instead of circular wires, as can be seen in FIG. 6. A spiral shape is considerably more flexible than fixed circular shapes. This flexibility may have advantages in terms of deployment in the smaller blood vessels, and across tortuous vasculature where deployment is more difficult and where the flow diverter stent 100 must have good flexibility to adopt the shape of the vasculature.

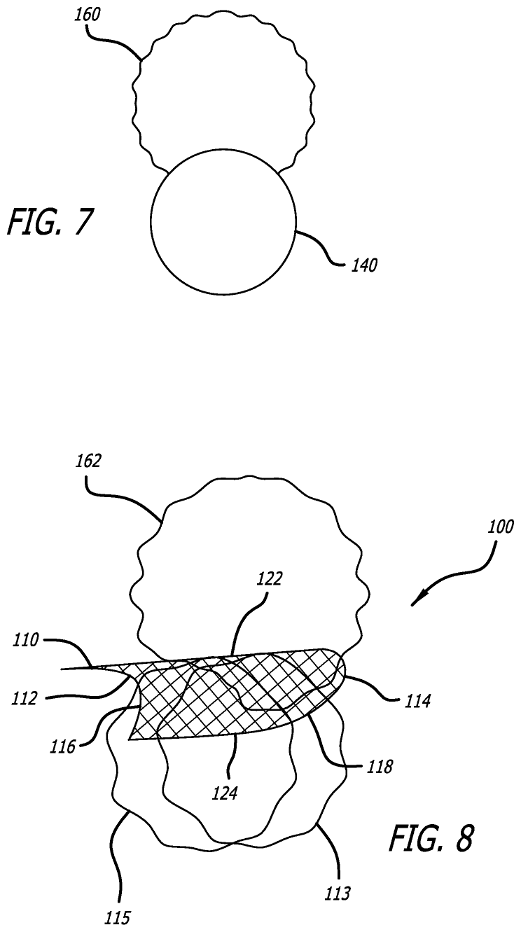

In yet some other embodiments, the proximal anchor 120 and distal anchor 140 may comprise a circular shape, however, the shape of the locator 160 may comprise a coiled or wavy wire as illustrated in FIG. 7.

According to some embodiments, as illustrated in FIG. 8, the proximal anchor 120 which includes but is not limited to a shape of a coiled wire and connects the proximal portion 113 and distal portion 115 of the flow diverter 110. The flow diverter 110 may comprise a second anchor 115 which includes but is not limited to the shape of a coiled wire and connects the proximal portion 113 and distal portion 115 at the back edge 124 of the flow diverter 110. In some embodiments, the locator 160 includes but is not limited to the shape of a coiled wire and connects the proximal portion 113 and distal portion 115 of the flow diverter 110 in the middle.

In some embodiments of the present invention, the circular shaped wires or the coiled end shaped wires of the proximal anchor 120, distal anchor 140 and locator 160 may comprise flexible wires so that they do not rupture the blood vessel and aneurysm wall when placed inside the blood vessel and aneurysm.

In some embodiments of the present invention, the proximal anchor 120, distal anchor 140 and locator 160 of the flow diverter 110 may comprise wire, for example, a nitinol wire. In one embodiment, metallic shape memory wires (e.g., nitinol, stainless steel, cobalt-chromium) are used as anchors and locator. In one preferred embodiment, nitinol is used. In one embodiment, drawn-filled tubing (DFT) utilizing a radiopaque (e.g., platinum or tantalum) core and an outer metallic (e.g., nitinol) jacket are used; these DFT elements appear similar to, and function similarly to, wires. In some embodiments, the shape memory wires like nitinol have the ability of to undergo deformation at one temperature, stay in their deformed shape, then recover their original, undeformed shape upon removal of the external load. This property of the shape memory materials helps the proximal anchor 120, distal anchor 140 and the locator 160 to expand from collapsed shapes to the expanded shapes when the flow-diverting implant 100 is pushed out of a delivery catheter inside the vessel and in the aneurysm.

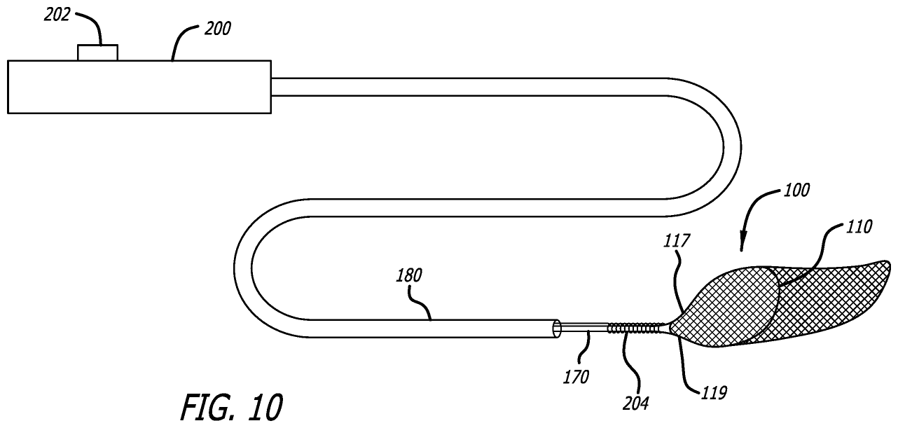

Referring now to FIGS. 9-13, there is shown a delivery pusher 170 used to deliver the flow diversion implant 100 to the target location. Pusher 170 is either a tubular or solid structure, which is gripped by a handle 200 at its proximal end by a user and used to push the connected implant 100 through an overlaying catheter 180 and to a treatment location. As shown in FIG. 10, the handle 200 includes a button 202 used to activate a detachment mechanism described below.

The delivery pusher 170 is releasably connected to the tapered corners 117, 119 of the implant 100 using a tether 204. Similar detachment mechanisms are shown and described in U.S. Pat. No. 8,182,506 to Fitz et al. entitled THERMAL DETACHMENT SYSTEM FOR IMPLANTABLE DEVICES and U.S. Pat. No. 8,597,323 to Plaza et al. entitled, DELIVERY AND DETACHMENT SYSTEMS AND METHODS FOR VASCULAR IMPLANTS, both of which are incorporated by reference herein.

The tether 204 may be a polymeric detachment tether/monofilament. A distal end of the tether 204 is wrapped around the tapered corners of the implant 100, while the proximal end of the tether 204 is surrounded by a heating mechanism exhibited by heater coil 210 and electrical leads 212. Electrical leads 212 run through a lumen of the pusher along with a core wire. Since the heater coil surrounds the detachment tether, heating of the heater coil results in severing of the tether 204 and subsequent detachment of the implant 100 from the pusher 170. Upon melting of the proximal end of the tether 204, the tether 204 unravels and the two proximal ends 117,119 of the implant separate to become positioned along the surface of the vessel lumen.

The pusher 170 functions to both push and rotate the flow-diverting implant 100 such that the flow diverter 110 can be properly placed at the neck of the aneurysm through radial expansion of the locator 160 inside the aneurysm. The proximal anchor 120, distal anchor 140 and locator 160 may optionally comprise radiopaque materials, for example, platinum or tantalum, to make their positions visible during delivery.

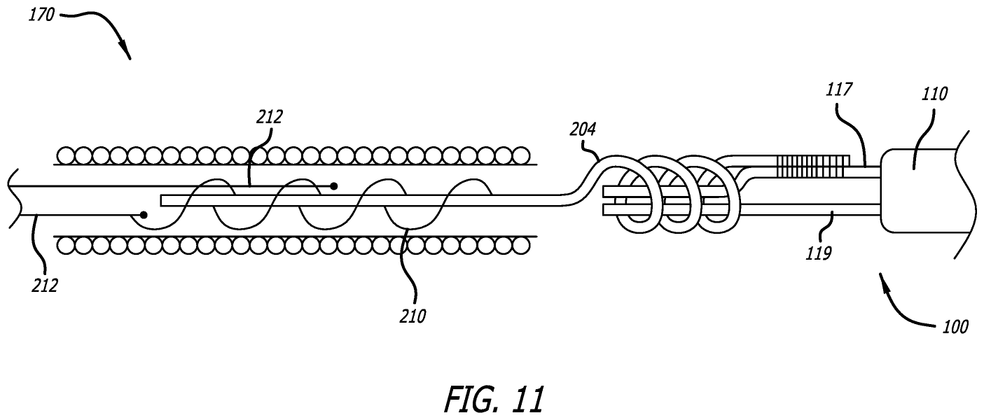

Although a heater 210 is depicted in FIG. 11, other embodiments of a detachment interface may utilize a mechanical connection, such as a screw where a user would simply rotate a screw-like engaging element in a particular direction to initiate detachment. Alternatively, an electrolytic system can be used where one wire is used to polarize a corrodible detachment junction and the patient's blood provides a return current to complete a detachment circuit. Electrolytic detachment systems are discussed in U.S. Pat. No. 5,122,136 to Guglielmi, entitled ENDOVASCULAR ELECTROLYTICALLY DETACHABLE GUIDEWIRE TIP FOR THE ELECTROFORMATION OF THROMBUS IN ARTERIES, VEINS, ANEURYSMS, VASCULAR MALFORMATIONS AND ARTERIOVENOUS FISTULAS, incorporated by reference in its entirety. Other detachment techniques generally known by one skilled in the art to detach an implant can be used to detach the implant 100 from pusher 170.

In one embodiment of the present invention, a method of blocking blood flow to an aneurysm includes navigating a delivery pusher containing an implant to a neck of an aneurysm. Next the implant is advanced out of the delivery pusher such that a locator unit of the implant expands into the aneurysm. The term “advanced” is meant to be interpreted relative to the delivery pusher. One skilled in the art will realize this may be accomplished by retracting an outer component relative to an inner component or by pushing an inner component forward relative to the outer component. As the implant continues to be advanced, a remaining portion of the implant is allowed to expand such that the flow diverter of the implant blocks the neck of the aneurysm and the anchor or anchors extends into the blood vessel from which the aneurysm formed. The location of the implant is verified. If acceptable, the button is depressed, closing the circuit to the heater, severing the tether and releasing the implant. If the location of the implant is not acceptable, the implant may be retracted back into the delivery device.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A flow diverting implant comprising:
a flow diverter;
a first anchor positioned at a proximal portion of the flow diverter;
a second anchor positioned at a distal portion of the flow diverter; and
a locator unit;
the locator unit configured such that when in a deployed state for reception in the aneurysm, the flow diverter is in a diverting state and the first and second anchors are in an anchoring state.

2. The flow diverting implant of claim 1, wherein the flow diverter is a single layer mesh structure.

3. The flow diverting implant of claim 1, wherein the flow diverter is woven from a single wire.

4. The flow diverting implant of claim 1, wherein the flow diverter comprises pores having sizes in a range of about 10-20 pores/mm².

5. The flow diverting implant of claim 1, wherein the flow diverter is slightly larger than the neck of the aneurysm.

6. The flow diverting implant of claim 1, wherein the flow diverter comprises a shape of a saddle

7. The flow diverting implant of claim 1, wherein the flow diverter has a rounded distal portion.

8. The flow diverting implant of claim 1, wherein the flow diverter has a semicircular proximal portion culminating in two tapered corners.

9. A flow diversion system comprising:
a delivery pusher including:
a proximal end and a distal end;
a heating element proximal the distal end;
a power source connected to the heating element with a switch;
an activation mechanism near the proximal end that closes the switch; an implant comprising:
a flow diverter having a proximal end;
at least one anchor attached to the flow diverter; and
a locator unit attached to the flow diverter;
wherein the implant is connected to the delivery pusher with a tether that passes within operable proximity to the heating element such that when the switch is closed, the tether is heated and severed, thereby releasing the implant.

10. The flow diversion system of claim 9 wherein the proximal end of the flow diverter is attached to the delivery pusher with the tether.

11. The flow diversion system of claim 10 wherein the proximal end of the flow diverter includes at least one tapered portion around which the tether is wrapped to connect the implant to the delivery pusher.

12. The flow diversion system of claim 11 wherein the at least one tapered portion comprises two tapered portions.

13. The flow diversion system of claim 12 wherein the two tapered portions are located at opposite sides of the proximal end of the flow diverter.

14. The flow diversion system of claim 9 wherein the at least one anchor comprises two anchors.

15. The flow diversion system of claim 14 wherein the locator unit is attached to one side of the flow diverter and the two anchor units are attached to an opposite side of the flow diverter.

16. The flow diversion system of claim 15 wherein the locator unit is attached near a center of the flow diverter and one of the two anchor units is attached proximal of the locator unit and the other of the two anchor units is attached distal of the locator unit.

17. A method of blocking blood flow to an aneurysm comprising:
navigating a delivery pusher containing an implant to a neck of an aneurysm;
advancing the implant out of the delivery pusher such that a locator unit of the implant expands into the aneurysm;
allowing a remaining portion of the implant to expand such that a flow diverter of the implant obstructs a neck of the aneurysm and at least one anchor extends into a blood vessel from which the aneurysm formed; and,
releasing the implant.

18. The method of claim 17, wherein advancing the implant out of the delivery pusher such that the locator unit of the implant expands into the aneurysm comprises rotating the pusher until the locator aligns with the neck of the aneurysm.

19. The method of claim 19 wherein releasing the implant comprises severing a tether connecting the implant to the delivery pusher.

20. The method of claim 19 wherein severing the tether comprises activating a heater in operable proximity to the tether.
